(19) United States
(12) Patent Application Publication
(10) Pub. No.: US 2005/0070818 A1
(43) Pub. Date: Mar. 31, 2005

(54) Biopsy device with viewing assembly

(76) Inventor: Richard L. Mueller JR., Jackson, WY (US)

Correspondence Address: OLSON & HIERL, LTD.;36th Floor, 20 North Wacker Drive, Chicago, IL, 60606, US

(21) Appl. No.: 10/675,459

(22) Filed: Sep. 30, 2003

(51) Int. Cl. A61B 10/00

(52) U.S. Cl. 600/564

(57) ABSTRACT

A biopsy device suitable for collection of a tissue sample from a biopsy site in a body lumen is provided. The biopsy device comprises a combination of an introducer assembly, a cutter assembly, and an endoscope assembly that coact with one another. The introducer assembly includes a hollow sheath having a distal end portion defining an aperture suitable for receiving a tissue mass therein. The cutter assembly includes a cutter tube having a distal end portion with a cutting edge. The cutter tube is sized to fit axially within the introducer sheath. The endoscope assembly includes a bundle of optical fibers sized to fit axially within the cutter tube. In use, the fiber optic bundle of the endoscope is nested within the cutter tube, which in turn is nested within the introducer sheath to form a co-axial structure.

### TECHNICAL FIELD OF THE INVENTION

The invention generally relates to a device for obtaining mammary duct tissue samples, such as a papilloma, and the like, for analysis. More specifically, the invention relates to a biopsy device having a viewing assembly and an introducer therefor.

### BACKGROUND OF THE INVENTION

Breast cancer is one of the health threats most feared by women, and is indeed the most common form of cancer in women. A key to treatment is early detection. For example, an annual mammogram is a method that has been used in hopes of early detection of breast cancer. One problem with mammography is that such an imaging technique can only find breast cancer once it has taken form. All too often, breast cancer is discovered at a stage that is too far advanced, when therapeutic options and survival rates are severely limited. While breast cancer is most common among women, in rare instances the human male may also have occurrences of breast cancer.

Other methods of detecting breast cancer are based on the fact that in a vast majority of instances breast cancer begins in the lining of mammary ducts. Studies have shown that fluid within the mammary duct contains high levels of breast cancer markers, and that an estimated 80%-90% of all breast cancers occur within the intraductal epithelium of the mammary glands. Fluid within the breast ducts contains an assemblage and concentration of hormones, growth factors and other potential markers comparable to those secreted by, or acting upon, the surrounding cells of the alveolar-ductal system. Likewise, mammary fluid typically contains cells and cellular debris or products that can also be used in cytological or immunological assays. As such, techniques such as ductal lavage, collection of mammary duct discharge, and brushing biopsies have been utilized to obtain such samples for diagnostic purposes.

While examination of mammary duct discharge has many benefits, one weakness is the relatively low and mixed cellularity of the specimens obtained. Therefore, in order to obtain targeted tissue samples for study, breast ductoscopy may be performed. This process includes passing an introducer past the sphincter muscle of a nipple orifice in a human breast and along the mammary duct. An endoscope is passed through the introducer to view the interior of the mammary duct. When a suspicious sample of tissue is viewed, such as a papilloma, a biopsy device can be introduced into the mammary duct, either via the introducer, or via a small incision in the breast to harvest the tissue sample for analysis.

One example of a biopsy device that is inserted through the breast tissue is the Mammotome® biopsy system available from Ethicon Endo-surgery, Inc., Cincinnati, Ohio. After mapping an area to be biopsied, the Mammotome® probe, a needle-like device with a hollow passage therethrough, is introduced through an incision cut into the breast and inserted with a sharpened distal end until the desired biopsy region is accessed. When the probe is positioned at the region of concern, tissue is received into a window in the probe with vacuum assist. A motor driven rotary cutter then cuts and removes tissue samples for examination. The samples are passed through the hollow passage of the probe into a collection chamber. Because the Mammotome® probe is directional, multiple specimens can be collected without having to remove and reinsert the device. The Mammotome® probe is removed after the samples have been collected, and the incision is closed.

Such a system significantly decreases the invasiveness of the biopsy procedure by only requiring a small incision and puncture, which may be done under local anesthetic. However, in certain situations, such as where the tissue to be biopsied is in a mammary duct, the incision and probe insertion required with the Mammotome® is unnecessarily invasive and undesirable.

What is needed is a biopsy device for conducting a minimally invasive biopsy procedure without the need for an incision or anesthetic. It is also desirable that the physician be able to take multiple tissue samples from a single biopsy site. It is also desirable for the physician to have the ability to inspect the region of the mammary duct that was biopsied to determine whether the desired tissue was completely excised or if additional samples should be taken. The present invention meets the foregoing desires and provides an improved device for taking a biopsy within a mammary duct.

### SUMMARY OF THE INVENTION

A biopsy device suitable for collection of a tissue sample from a biopsy site in a body lumen is provided. The biopsy device comprises an introducer assembly, a cutter assembly within the introducer assembly, and an endoscope assembly enveloped by the cutter assembly.

The introducer assembly includes a hollow sheath having a distal end portion and a proximal end portion. The distal end portion of the introducer sheath defines an aperture suitable for receiving a tissue mass therein. The cutter assembly also includes a hollow sheath having a distal end portion and a proximal end portion, but an outside diameter less than the inside diameter of the introducer hollow sheath. The cutter sheath is sized to fit axially within the introducer sheath. The endoscope assembly includes a bundle of optical fibers for viewing and illuminating the biopsy site. The bundle is sized to fit axially within the cutter sheath. In use, the fiber optic bundle of the endoscope is nested within the cutter sheath, which in turn is nested within the introducer sheath to form a tri-axial structure comprising three sheathes or tubes.

The cutter sheath and introducer sheath distal end portions are moved relative to one another so as to cause a tissue sample that extends into the biopsy device though the introducer sheath aperture to be scissored or cut from the mammary duct wall. It is not critical to the present device whether the cutter sheath or the introducer sheath is movable as long as relative movement between these two structural components effects cutting of the tissue. In other words, movement of the cutter sheath, the introducer sheath, or a combined movement of the two may cut the tissue. During the cutting process, the viewing end of the endoscope is positionable near the aperture through which the tissue extends such that the cutting procedure may be viewed by a practitioner as it takes place.

### BRIEF DESCRIPTION OF THE DRAWINGS

In the drawings,

FIG. 1 is an exploded side view of a biopsy device embodying the present invention;

FIG. 2 is an enlarged partial perspective view of the distal end portion of the biopsy device shown in FIG. 1;

FIG. 3 is a side view of the biopsy device in a human breast shown in section;

FIG. 4 is the biopsy device of FIG. 3 before cutting a papilloma;

FIG. 5 is an exploded side view of a biopsy device that depicts an alternate embodiment of the present invention with a cutter assembly suitable for coring tissue;

FIG. 6 is an enlarged partial perspective view of the biopsy device of FIG. 5 with the cutter in a pre-cut position;

FIG. 7 is an enlarged partial cross sectional side view of the distal end portion of the biopsy device of FIG. 5 after cutting a tissue sample;

FIG. 8 is an enlarged partial cross sectional side view of the biopsy device of FIG. 5 coring an occlusion in a mammary duct;

FIG. 9 is an enlarged partial cross sectional perspective view of the distal end portion of a biopsy device that illustrates yet another embodiment of the present invention;

FIG. 10 is an enlarged partial cross sectional perspective view of the distal end portion of another biopsy device that illustrates s still further embodiment of the present invention; and

FIG. 11, is an enlarged partial perspective view of the distal end portion of yet another biopsy device that illustrates an embodiment of the present invention.

### DESCRIPTION OF THE PREFERRED EMBODIMENTS OF THE INVENTION

The invention disclosed herein is susceptible of embodiment in many different forms. Shown in the drawings and described hereinbelow in detail are preferred embodiments of the invention. It is to be understood, however, that the present disclosure is an exemplification of the principles of the invention and does not limit the invention to the illustrated embodiments.

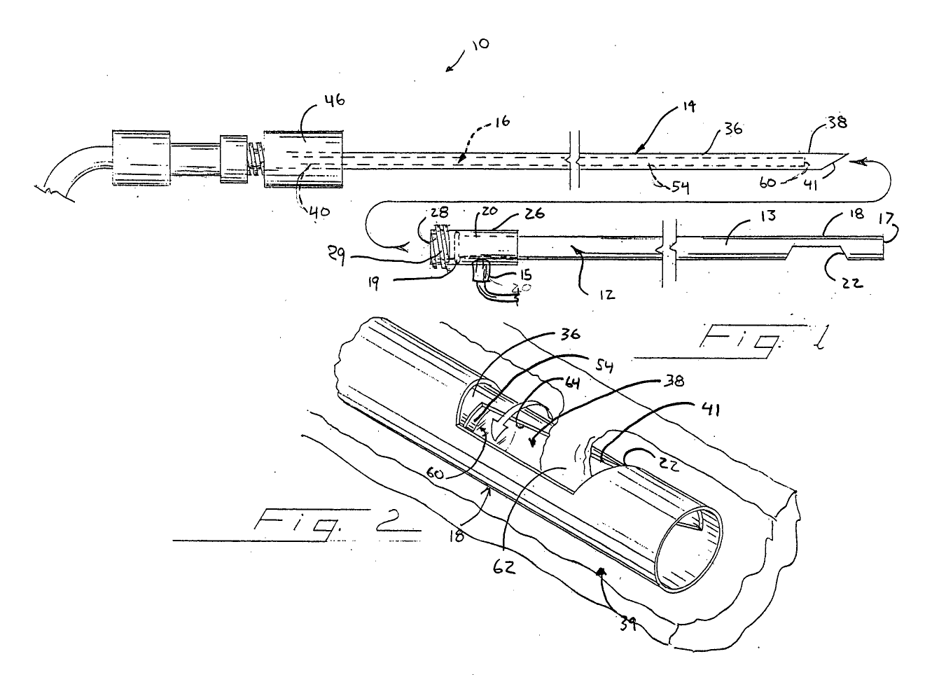

The biopsy device shown in FIG. 1 represents one preferred embodiment of the present invention. Biopsy device 10 is suitable for collection of a tissue sample from a biopsy site in a body lumen and is comprised of an introducer assembly 12, a cutter assembly 14 receivable within introducer assembly 12, and an endoscope assembly 16 shown in phantom within cutter assembly 14.

The introducer assembly 12 includes a hollow sheath 13 having a distal end portion 18 and a proximal end portion 20 provided with external threads 29. The sheath distal end portion 18 includes an aperture 22 that is suitable for receiving a tissue mass therethrough. The introducer assembly 12 may further include an atraumatic tip, e.g., a rounded edge, polyflourocarbon coated tip, or the like, on the distal end of introducer sheath 13. The sheath 13 is also preferably made of a relatively rigid material, such as stainless steel. Provided about and secured to the introducer sheath proximal end portion 20 is an introducer handle 26. The introducer handle 26 includes a passage 28 which is in communication with the sheath 13. Preferably, passage 28 is tapered so as to enhance the ability to insert the cutter assembly 14 into introducer sheath 13. Handle assembly 26 may also include an irrigation or vacuum flush port 15, if desired. A seal, such as o-ring 19, may be included to seal component parts such that liquid entering or exiting port 15 is directed to or from aperture 22 or distal end 17 of the introducer sheath 13. The handle assembly 26 is preferably made of a rigid material, such as a hard plastic. In this embodiment, the handle assembly 26 and the sheath 13 are shown as being integral components, however, they may be unitary with one another as well.

The cutter assembly 14 comprises a hollow cutter tube or cylinder 36 having a distal end portion 38 and a proximal end portion 40. The cutter tube 36 is slidably received within and sized to extend axially through the introducer sheath 13. The cutter tube 36 is extendable and retractable within the introducer sheath 13. The relative diameters of the cutter sheath 36 and introducer sheath 13 are such that the cutter sheath 36 can be rotated or axially moved within the introducer sheath 13. The inner diameter of the introducer sheath 13 is preferably less than about 0.001 inches greater than the outer diameter of the cutter tube 36, and more preferably about 0.0002 inches and 0.005 inches greater than the outer diameter of the cutter tube. The relatively tight tolerances enhance the cutting or scissoring effect between the cutter sheath 36 and the introducer sheath 13 as they move relative to each other. The distal end portion 38 of the cutter tube 36 includes a notch 41 in this particular embodiment. Notch 41 is preferably larger than the side aperture 22 in the introducer sheath 13. As will be explained below in further detail, the notch 41, in cooperation with side aperture 22, excises tissue that extends through aperture 22. In this embodiment, the proximal end portion 40 of cutter sheath 36 terminates in internally threaded holster 46 which also threadedly engages external threads 29 on introducer sheath proximal end portion 20.

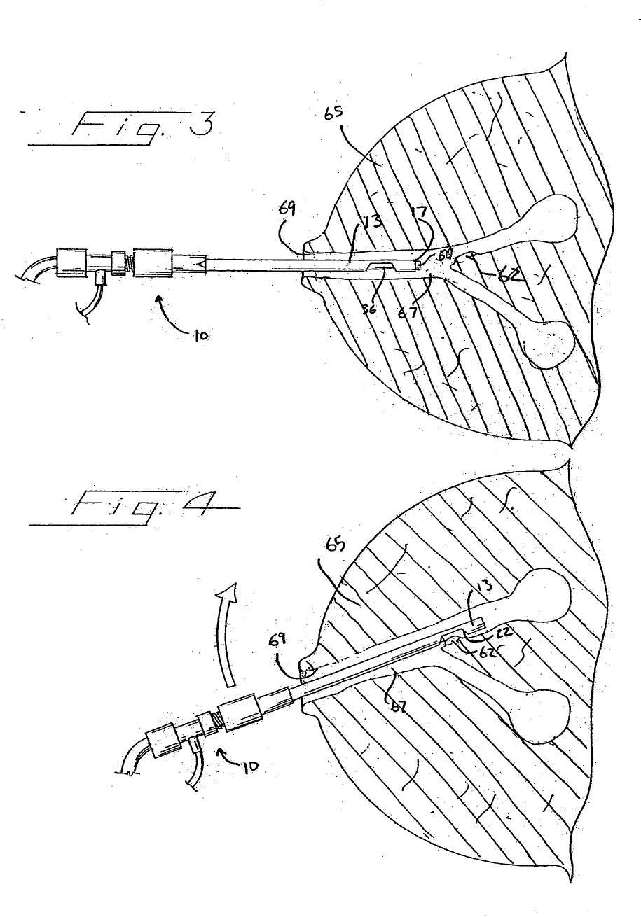

Endoscope assembly 16 includes a elongated fiber optic bundle 54 comprising optical fibers for illumination and viewing of the biopsy region. The bundle 54 terminates at a endoscope viewing end 60. Elongated fiber optic bundle 54 is sized to be received axially though the cutter sheath 36. The difference between the outer diameter of endoscope bundle 54 and the inner diameter of cutter tube 36 is preferably about 0.002 inches to 0.005 inches. Thus, endoscope bundle 54 and cutter tube 35 together define a passageway for the introduction or removal of liquid, such as a saline or an anaesthetic, such as a lidocaine flushing solution. In use, the endoscope viewing end portion 60 is positioned about the distal end portion 38 of cutter tube 36, desirably is adjacent to notch 41, and together form a working end portion 39 (FIG. 2) of the biopsy device. As such, a practitioner can view, via the endoscope assembly 16 the distal end portion 38 of cutter tube 36, which in use, is positioned at the aperture 22 of introducer sheath 13.

The cutting of the target tissue sample at working end portion 39 is shown in FIG. 2. The viewing end 60 of the bundle 54 of the endoscope assembly 16 is positioned adjacent to notch 41 of the cutter tube 36. Prior to cutting the papilloma 62, notch 41 is positioned such that cutter tube 36 does not occlude aperture 22 of introducer sheath 18. Target tissue, such as papilloma 62, is passed through aperture 22. This may be accomplished by manipulating biopsy device 10, or by manipulating the patient's breast itself, or a combination thereof. When the target tissue is positioned within the distal end portion 18 of the introducer and the distal end portion 38 of the cutter tube 36, cutter tube 36 is rotated such that notch 41 is rotated relative to aperture 22. As the edge 64 of notch 41 is rotated and as papilloma 62 is pinched between edge 64 and aperture 22, edge 64 and aperture 22 sever the papilloma 62. Generally, the thickness of the cutter tube 36 is relatively thin and is suitable for cutting the tissue, however, it may be preferable for edge 64 to be sharpened to enhance the cutting ability. The need for sharpening is modest as the majority of papillomas and ductal protrusions are loosely attached and do not require a very sharp edge to cut. The endoscope assembly 16, cutter assembly 14 (FIG. 1) and severed papilloma 62 can then be withdrawn from the introducer assembly 12 and the tissue harvested for analysis. The distal end portion 18 of sheath 13 of the introducer assembly 12 can be left at the biopsy site to mark its location. The endoscope assembly 16 and cutter assembly 14 can then be reinserted into the introducer assembly 12 to view the biopsy site to determine whether the entire papilloma was removed, whether the excision was taken at the correct location, and to view the cut surfaces. The practitioner may also be able to view other tissue samples for excision in the same region of the duct.

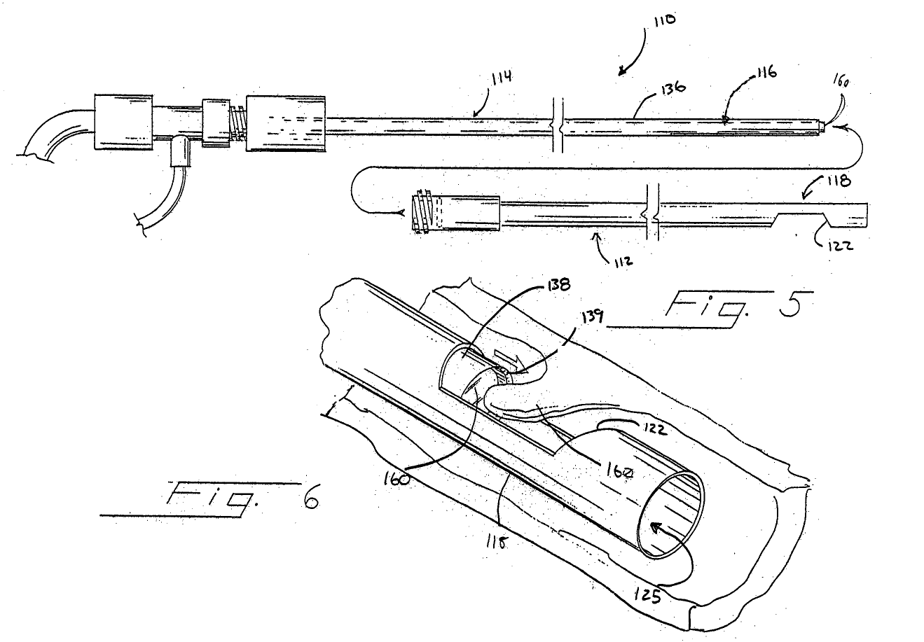

A preferred method of using the biopsy device is described with respect to FIGS. 3 and 4. The biopsy device 10 from FIG. 1 is utilized in this example. A human breast 65 typically includes mammary ducts, such as mammary duct 67, which terminate at nipple orifices such as orifice 69 at the nipple surface and extend approximately 1 to 4 inches into the breast 65 branching several times. The nipple surface often includes 8 to 12 separate nipple orifices. In order to obtain a tissue sample with the biopsy device 10, the introducer sheath 13 is inserted into a mammary duct 67 via a nipple orifice 69. The desired nipple orifice is first located through use of any means such as an illuminated nipple cup (not shown). A nipple orifice dilator or catheter (not shown) may be used to dilate the nipple orifice, if desired, to permit easier insertion of the introducer sheath 13. Cutter tube 36 and the fiber optic bundle are extended from within the introducer sheath 13 to enable viewing of the biopsy site. Preferably, the distal viewing end 60 of the fiber optic bundle is positioned to be coterminus with or extended slightly beyond the distal end 17 of the introducer sheath 13 so that a practitioner can view the mammary duct 67 as the biopsy device 10 is guided therein. When a target mass of tissue is located, such as papilloma 62, the endoscope fiber optic bundle 54 is retracted such that the endoscope viewing end 60 is positioned at the proximal end of aperture 22 in the introducer sheath 13.

The tissue mass is received within the sheath 26 through the aperture or cut-out 22. As discussed, the tissue mass can be urged into the aperture 22 by external pressure applied to the breast. Alternatively a vacuum source may be associated with the introducer sheath to assist in urging the tissue into the aperture. As discussed above with respect to FIG. 2, the cutter tube 36 is rotated such that edge 64 of notch 41 travels across aperture 22, thereby severing a tissue sample. The entire biopsy device 10 can then be removed from the mammary duct together with the sample contained therewithin. Alternatively, only the cutter assembly 14 with tissue sample and endoscope assembly 16 therein can be removed, and the distal end portion 18 of the introducer sheath 13 left at the biopsy site for additional inspection or more sampling. One advantage of the present invention is that the biopsy site is marked by the distal end portion 18 of the introducer sheath 13 and does not require a separate marking device or marking step. If desired, a separate marking device, such as a detachable anchor (not shown) may be included in the introducer sheath to mark the biopsy site for later patient visits.

In the illustrative embodiments that follow, the last two digits of the numerals denote features that are the same or similar in function to the features described hereinabove.

An alternate embodiment of the present invention is shown in FIGS. 5-7. Similar to the embodiment in FIGS. 1-3, a biopsy device 110 includes an introducer assembly 112, a cutter assembly 114 and an endoscope assembly 116.

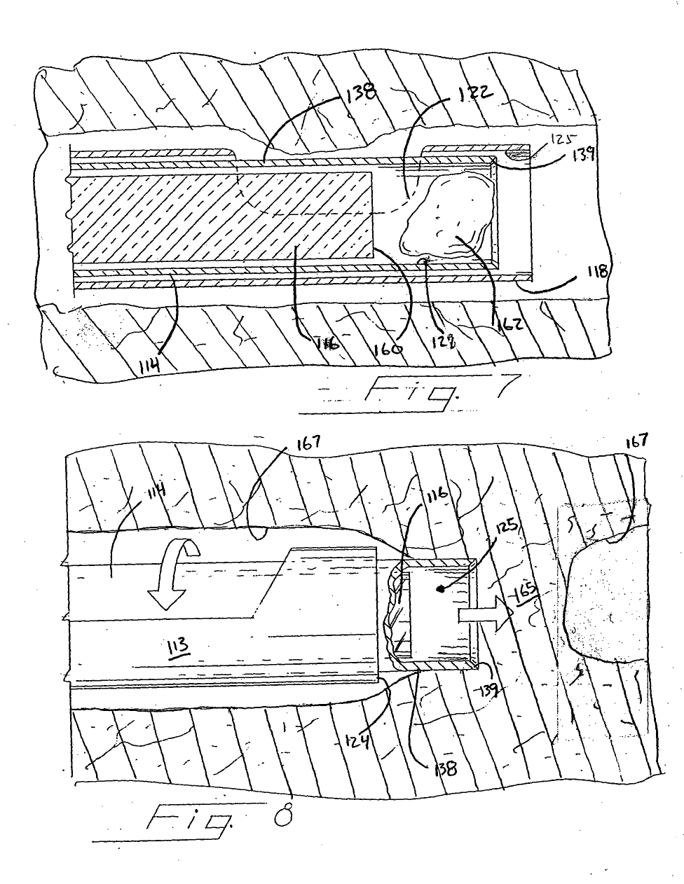

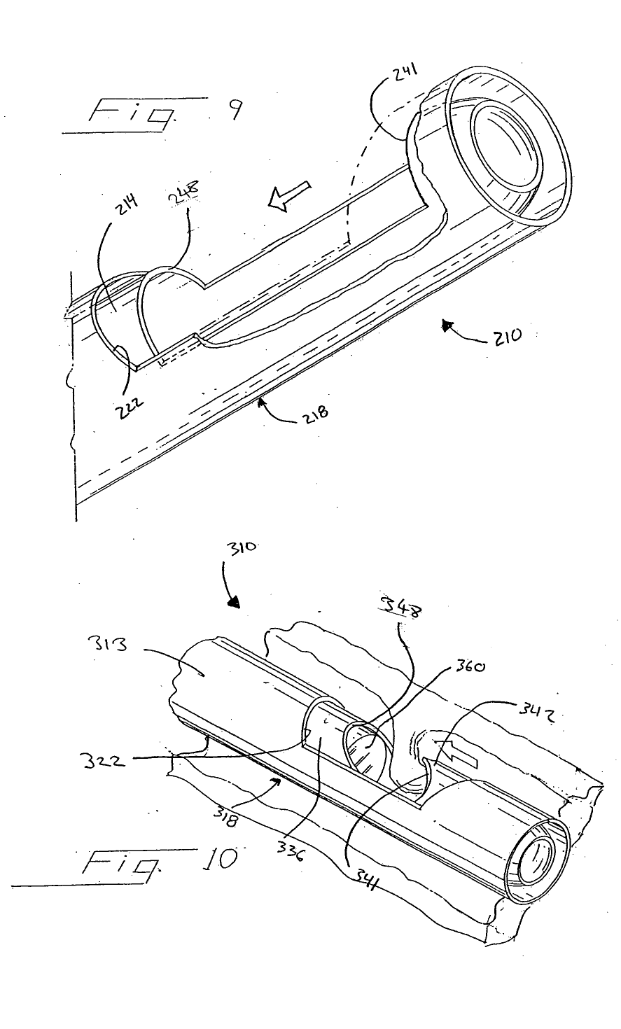

The introducer assembly 112 and the endoscope assembly 116 are substantially the same as in the embodiment shown in FIGS. 1-3. The cutter assembly 114, however, is different. Referring to FIG. 6, the distal end portion 138 of cutter cylinder 136 does not include a notch. Instead, the distal end 139 of the cutter cylinder 136 is a cutting edge. As cutter cylinder 136 is moved axially towards the distal end portion 118 of introducer sheath 113 and passed across side aperture 122, through which tissue, such as papilloma 162 extends, distal end 139 severs a tissue sample therefrom. Side aperture 122 is spaced away from the distal end 124 of introducer 112 so as to provide an introducer reservoir 125 for retaining the tissue sample The severed tissue may be collected in reservoir 125 as shown in FIG. 7. Preferably, endoscope viewing end 160 is positioned proximal to cutter distal end 139 so as to provide an additional cutter reservoir 127 in addition to introducer reservoir 125. This embodiment may also be used in scraping procedures since the cutter distal end 139 travels across the entire aperture 122 of introducer 112.

This particular embodiment also has the ability to core blockages or occlusions in ductal systems. For example, as shown in FIG. 8, a blockage 165 is shown in duct 167. The distal end portion 138 of the cutter cylinder 136 is extended beyond the distal end 124 of the introducer sheath 113. The distal end 139 of the cutter cylinder 136 can be pushed through the blockage or can be rotated into the blockage or occlusion 165 to core out tissue. The cutter assembly 114 and endoscope assembly 116 can be withdrawn from the introducer and the tissue sample removed. The normally loose fibrous nature of invasive duct tissues allows a simple coring cutter to sever tissue in the forward cutting mode without concurrent shearing by the introducer sheath 113. The same process may be repeated as desired, and may result in a clearance of the blockage. In order to enhance the retention of tissue samples within the biopsy device, the distal introducer reservoir 125 can include a textured interior such as by way of a fine thread or sand blasted surface.

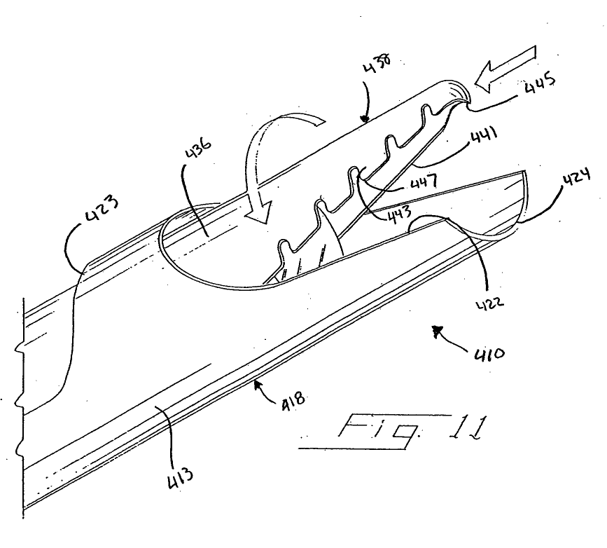

FIG. 9 shows another embodiment. The biopsy device 210 is provided with a different cutter assembly 214. At the distal end portion 238 of cutter tube 236 is formed a cutter side aperture 258 preferably configured to cover approximately the same area as side aperture 222 formed in the distal end portion 218 of introducer sheath 213. In use, side aperture 222 and cutter side aperture 248 are aligned with one another. After the target tissue, such as papilloma 262, enters through aperture 222 and cutter side aperture 248, cutter tube 236 is moved proximally such that edge 241 of cutter tube 236 severs the tissue. In an alternate embodiment of the device 310 that is shown in FIG. 10, the cutter side aperture 348 is provided with a barbed or hook section 342 which acts to grasp the tissue to be cut by edge 341 delineating cutter tube aperture 348 as the cutter tube 336 is moved proximally across side aperture 322. During the cutting procedure, endoscope viewing end 360 is positioned adjacent and proximal to the side aperture 322 of the introducer sheath 313 so as to enable the practitioner to view the biopsy site during the procedure.

Thus far, the embodiments provided have all included a side aperture which is spaced from the distal end of the introducer sheath. Shown in FIG. 11 is an embodiment of the device 410 where a side aperture is not spaced from the distal end of the introducer sheath, but instead, is complementary with the distal end. In this particular embodiment, introducer sheath 413 includes a distal end portion 418 having side aperture 422, which is complementary with distal end 424. The distal end portion 438 of the cutter tube 436, in this embodiment, includes a notch 441. Similar to previous embodiments, the notch 441, in cooperation with aperture 422, excises the desired tissue that is extended through aperture 422. For example, as cutter tube 436 is rotated, the scissoring interaction between cutter tube 436 about notch 441 and aperture 422 severs the tissue. Additional features that may be included in the notch 441.

For example, notch 441 optionally includes crenations 443 and a cusp 445 at the distal end of cutter tube 436. The crenations 443 enhance the cutting operation and also serve to hold the tissue in place as the cutter tube 436 is rotated relative to the introducer sheath 413. Preferably, crenations 443 are angled towards the proximal end, thereby creating a series of points 447 to further enhance cutting. The cusp 445 may be used to assist in retaining the severed tissue inside the cutter tube 436 as it is withdrawn from the introducer sheath 413.

Also shown in this embodiment is the use of a plurality of apertures in the distal end portion 418 of the introducer sheath 413. In addition to side aperture 422, aperture 423 is also suitable for receiving target tissue therethrough. Cutter tube 436 is suitable for severing tissue either received through aperture 423 or into aperture 422.

The foregoing descriptions are to be taken as illustrative, but not limiting. Still other variants within the spirit and scope of the present invention will readily present themselves to those skilled in the art.

1. A biopsy device suitable for collection of a tissue sample from a biopsy site in a body lumen, the biopsy device comprising:
an introducer assembly comprising a hollow sheath having a distal end portion and a proximal end portion, and the distal end portion of the introducer sheath defining at least one side aperture for receiving a tissue mass;
a cutter assembly comprising a hollow cutter tube having a distal end portion and a proximal end portion, the cutter tube being sized to be slidable received within and extend axially through the introducer sheath, and the distal end portion of the cutter tube including at least one cutting edge;
an endoscope assembly including a fiber optic bundle for viewing the biopsy site, the bundle being sized to be received within and extend axially though the cutter tube; and
the cutter tube and the introducer sheath cooperating to sever the tissue sample by relative movement therebetween.

2. The biopsy device of claim 1, wherein the distal end portion of the cutter tube further defines a notch.

3. The biopsy device of claim 2, wherein the distal end portion of the cutter tube is rotatable relative to the introducer distal end portion.

4. The biopsy device of claim 2, wherein the introducer distal end portion is rotatable relative to the distal end portion of the cutter tube.

5. The biopsy device of claim 1, wherein the distal end portion of the cutter tube includes a cutter aperture.

6. The biopsy device of claim 5, wherein the distal end portion of the cutter tube is rotatable relative to the introducer distal end portion.

7. The biopsy device of claim 5, wherein the introducer distal end portion is rotatable relative to the distal end portion of the cutter tube.

8. The biopsy device of claim 5, wherein the distal end portion of the cutter tube is axially reciprocatable relative to the introducer distal end portion.

9. The biopsy device of claim 5, wherein the cutter aperture further includes a barbed portion.

10. The biopsy device of claim 1, wherein the distal end portion of the cutter tube includes a cutting edge.

11. The biopsy device of claim 10, wherein the distal end portion of the cutter tube is extendable past the distal end of the introducer.

12. The biopsy device of claim 11, wherein the cutter tube is rotatable.

13. The biopsy device of claim 10, wherein the introducer sheath at the distal end portion thereof defines an introducer reservoir.

14. The biopsy device of claim 1, wherein the cutter tube and the endoscope assembly define a liquid passageway.

15. The biopsy device of claim 1, wherein the introducer hollow sheath inner diameter is about 0.001 inches to about 0.1 inches greater than the outer diameter of the cutter tube.

16. The biopsy device of claim 1, wherein the introducer hollow sheath inner diameter is about 0.0002 inches to about 0.001 inches greater than the outer diameter of the cutter tube.

17. The biopsy device of claim 1, wherein the introducer side aperture has an axial length of about 1 millimeter to about 5 millimeters.

18. The biopsy device of claim 1, wherein the introducer side aperture has an axial length of about 2 millimeter to about 3 millimeters.

19. The biopsy device of claim 1, wherein the introducer side aperture extends over about one-half the introducer hollow sheath circumference.

20. The biopsy device of claim 1, wherein the introducer distal end portion includes an open end.

21. The biopsy device of claim 1, wherein the introducer distal end portion includes a plurality of side apertures.

22. The biopsy device of claim 1, wherein the cutting edge is provided with crenations.

23. A biopsy device suitable for collection of a tissue sample from a biopsy site in a body lumen, the biopsy device comprising:
an introducer assembly, a cutter assembly slidably received within the introducer assembly, and an endoscope assembly situated within the cutter assembly;
the introducer assembly comprising a tubular sheath having a distal end portion that defines a side aperture for receiving the tissue sample;
the cutter assembly comprising a hollow cylindrical cutter having a distal end portion, the cutter being co-axial with the introducer sheath and having a lesser outside diameter than the introducer sheath inside diameter, the cylindrical cutter including at least one cutting edge;
the endoscope assembly comprising a fiber optic bundle, the fiber optic bundle being co-axial with the hollow cutter and having a lesser outside diameter than the cutter inside diameter; and
at least the introducer distal end portion and the cutter distal end portion being mounted for movement relative to one another, the relative movement of the cutter distal end portion and the distal end portion of the tubular sheath causing the cutting of the tissue sample received therebetween.

24. The biopsy device of claim 23, wherein the distal end portion of the cylindrical cutter further defines a notch.

25. The biopsy device of claim 24, wherein the distal end portion of the cylindrical cutter is rotatable relative to the sheath distal end portion.

26. The biopsy device of claim 24, wherein the sheath distal end portion is rotatable relative to the distal end portion of the cylindrical cutter.

27. The biopsy device of claim 23, wherein the distal end portion of the cylindrical cutter includes a cutter aperture.

28. The biopsy device of claim 27, wherein the distal end portion of the cylindrical cutter is rotatable relative to the sheath distal end portion.

29. The biopsy device of claim 27, wherein the sheath distal end portion is rotatable relative to the distal end portion of the cylindrical cutter.

30. The biopsy device of claim 27, wherein the distal end portion of the cylindrical cutter is axially reciprocatable relative to the sheath distal end portion.

31. The biopsy device of claim 27, wherein the cutter aperture further includes a barbed portion.

32. The biopsy device of claim 23, wherein the distal end portion of the cylindrical cutter includes a cutting edge.

33. The biopsy device of claim 32, wherein the distal end portion of the cylindrical cutter is extendable past the distal end of the sheath.

34. The biopsy device of claim 33, wherein the cylindrical cutter is rotatable.

35. The biopsy device of claim 32, wherein the sheath at the distal end portion thereof defines an introducer reservoir.

36. The biopsy device of claim 23, wherein the cylindrical cutter and the endoscope assembly define a liquid passageway.

37. The biopsy device of claim 23, wherein the sheath inner diameter is about 0.001 inches to about 0.1 inches greater than the outer diameter of the cylindrical cutter.

38. The biopsy device of claim 23, wherein the sheath inner diameter is about 0.0002 inches to about 0.001 inches greater than the outer diameter of the cylindrical cutter.

39. The biopsy device of claim 23, wherein the introducer side aperture has an axial length of about 1 millimeter to about 5 millimeters.

40. The biopsy device of claim 23, wherein the introducer side aperture has an axial length of about 2 millimeter to about 3 millimeters.

41. The biopsy device of claim 23, wherein the introducer side aperture extends over about one-half the sheath circumference.

42. The biopsy device of claim 23, wherein the sheath distal end portion includes an open end.

43. The biopsy device of claim 23, wherein the sheath distal end portion includes a plurality of side apertures.

44. The biopsy device of claim 23, wherein the cutting edge is provided with crenations.

45. A biopsy device suitable for collection of a tissue sample from a biopsy site in a body lumen, the biopsy device comprising an introducer assembly having an introducer distal end portion, a cutter assembly within the introducer assembly and having a cutter distal end portion, and an endoscope within the cutter assembly and having a fiber optic bundle distal end portion;
a working end portion of the biopsy device comprising the introducer distal end portion, the cutter distal end portion, and the fiber optic bundle distal end portion;
the introducer distal end portion having a tubular configuration and defining at least one side aperture;
the cutter distal end portion having a tubular configuration and including a cutting edge;
the cutter distal end portion being slidably received within the introducer distal end portion and the fiber optic bundle distal end portion being received inserted within the cutter distal end portion, and
the cutter distal end portion coacting with the introducer distal end portion to cut the tissue sample.

46. The biopsy device of claim 45, wherein the cutter distal end portion further defines a notch.

47. The biopsy device of claim 46, wherein the cutter distal end portion is rotatable relative to the introducer distal end portion.

48. The biopsy device of claim 46, wherein the introducer distal end portion is rotatable relative to the cutter distal end portion.

49. The biopsy device of claim 45, wherein the cutter distal end portion further includes a cutter aperture.

50. The biopsy device of claim 49, wherein the cutter distal end portion is rotatable relative to the introducer distal end portion.

51. The biopsy device of claim 49, wherein the introducer distal end portion is rotatable relative to the cutter distal end portion.

52. The biopsy device of claim 49, wherein the cutter distal end portion is axially reciprocatable relative to the introducer distal end portion.

53. The biopsy device of claim 49, wherein the cutter aperture further includes a barbed portion.

54. The biopsy device of claim 45, wherein the cutter distal end portion includes a cutting edge.

55. The biopsy device of claim 54, wherein the cutter distal end portion is extendable past the introducer distal end portion.

56. The biopsy device of claim 55, wherein the cutter distal end portion is rotatable.

57. The biopsy device of claim 54, wherein the introducer distal end portion includes an introducer reservoir.

58. The biopsy device of claim 45, wherein the cutter distal end portion and the endoscope assembly define a passageway.

59. The biopsy device of claim 45, wherein the introducer distal end portion is about 0.001 inches to about 0.1 inches greater less than the outer diameter of the cutter distal end portion.

60. The biopsy device of claim 45, wherein the introducer distal end portion has an inner diameter about 0.0002 inches to about 0.001 inches greater than the outer diameter of the cutter distal end portion.

61. The biopsy device of claim 45, wherein the introducer side aperture has an axial length of about 1 millimeter to about 5 millimeters.

62. The biopsy device of claim 45, wherein the introducer side aperture has an axial length of about 2 millimeter to about 3 millimeters.

63. The biopsy device of claim 45, wherein the introducer side aperture extends over about one-half the introducer distal end portion circumference.

64. The biopsy device of claim 45, wherein the introducer distal end portion includes an open end.

65. The biopsy device of claim 45, wherein the introducer distal end portion includes a plurality of side apertures.

66. The biopsy device of claim 45, wherein the cutting edge further includes crenations.
